(19) United States
(12) Patent Application Publication
(10) Pub. No.: US 2013/0045481 A1
(43) Pub. Date: Feb. 21, 2013

(54) PROCESS FOR THE IDENTIFICATION AND TRACEABILITY OF PLANT COMPONENTS

(71) Applicants: Diego Breviario, Milan (IT); Anna Paola Casazza, Milan (IT); Floriana Gavazzi, Milan (IT); Elena Ponzoni, Milan (IT); Luca Braglia, Milan (IT); Silvia Giani', Milan (IT)

(72) Inventors: Diego Breviario, Milan (IT); Anna Paola Casazza, Milan (IT); Floriana Gavazzi, Milan (IT); Elena Ponzoni, Milan (IT); Luca Braglia, Milan (IT); Silvia Giani', Milan (IT)

(21) Appl. No.: 13/577,046

(22) PCT Filed: Jan. 31, 2011
(86) PCT No.: PCT/IB11/00146

§ 371 Date: Oct. 23, 2012

(30) Foreign Application Priority Data
Feb. 3, 2010 (IT) MI2010A0001633

(51) Int. Cl. C12Q 1/68; C07H 21/04

(52) U.S. Cl. 435/6.12; 536/24.33

(57) ABSTRACT

The invention concerns the field of plant genomics and in particular the field of molecular diagnosis. In particular, the invention refers to a process for the identification of plant species and varieties, which can be identified both individually and in a mixture. The invention also concerns a kit for recognition of the different plant species and varieties.

### TECHNICAL BACKGROUND

For different reasons connected with health, certification of product quality standards and speculative research, it is necessary to verify the presence, or absence, of distinct plant varieties and species.

For example, food for human consumption defined as “organic” or ayurvedic foodstuffs, or products in the agri-food sector distinguished by a certification mark (i.e. doc, docg, pgi, tgi, pdo, tsg) required by the current European laws to comply with standards of quality and composition.

In order to safeguard the traditional enogastronomic products of each country, the European Union has issued regulations which, since 1992, have established the requirements and procedures to obtain a designation and a mark of quality, to ensure their origin and connection with a particular area, protecting them from imitations and fraud by competitors. The certifications are therefore an important guarantee for the consumer, as they represent a declaration, by an Organisation, that that particular foodstuff or drink complies with certain production specifications and specific quality standards.

The food certifications known to the public as quality marks recognised by the European Union define the following products:
PDO—Protected Designation of Origin (typical product which is inseparably linked to its area of origin, where the entire production process takes place, from the raw materials to transformation)
PGI—Protected Geographical Indication (typical product which is not so closely linked to its area. The raw materials may not be local, but at least one stage of the production process must be performed in the specific area)
TSG—Traditional Speciality Guaranteed (a product with characteristics that do not depend on the place of origin, but with specificity linked to a recipe, a composition or a traditional method)

For a product to be eligible for one of the above-mentioned certifications, the producer must therefore comply with detailed and meticulous specifications as occurs, for example, in the case of one of the best-known Italian products: Parmigiano Reggiano. In the case of Parmigiano Reggiano, the cattle feed used must not contain any sugar beet, fenugreek, rice or rape.

In this regard, the importance of a method able to highlight the presence, or verify the absence, of certain specific plant species is evident. Said method is particularly useful in determining the composition of animal feed both because it permits verification of the actual content, guaranteeing a good state of health of the animal and an appropriate nutritional intake, but also because the feed is eaten by animals the by-products of which are consumed by humans. For example, finished or semi-finished foodstuffs such as meat, milk or its by-products.

Furthermore, a constantly increasing number of people follow a medicine defined as “alternative” or “non-conventional”, in which the diet is based on phytoextracts and many different elements of plant origin. Said products or preparations commonly known as “natural” are now consumed extensively, especially in the treatment of states or syndromes attributable to lifestyle. It is therefore necessary to be able to verify their actual composition since they are intended for consumption by humans.

### OBJECTS OF THE INVENTION

The object of the present invention is to make available a process for the identification of plant species and varieties that can be identified either individually or in mixtures, coming from pasture areas, parks and gardens, products of plant origin intended for feeding animals (feed and fodder) and humans (agri-food sector products).

A further object of the present invention is to make available a process for monitoring the composition in terms of plant species and varieties of diets used for feeding animals, the products and by-products of which are distinguished by marks, the quality certification and geographical origin of which depend on production specifications which require the presence of precise plant species and the absence of others.

A further object of the invention is a process for the recognition of contaminations of plant origin in products intended for animal and human consumption.

A further object of the present invention is to make available a process for the traceability and re-traceability of individual plant components in the production chain: animal diet (grass from pastures-feed)-milk-cheese.

A further object of the present invention is to make available a process for identifying and monitoring plant species and varieties present in composite mixtures which is rapid, inexpensive, accurate, reproducible and reliable on a wide scale.

A further object of the present invention is to make available a kit for the recognition and traceability of plant components, identifiable both individually and in a mixture, whether they come from pasture areas, parks and gardens or are present in feed and products in the agri-food sector intended for animal and human consumption.

### DESCRIPTION OF THE FIGURES

FIG. 1 shows the selective amplification of the intron sequences of β-tubulin genes of wheat (F), maize (M), barley (O) and soybeans (S).

FIG. 2 shows the result of specific amplification assays of the intron regions to verify specificity and selectivity with respect to the species of the probes designed on the introns of wheat, maize, barley and soybeans.

### DESCRIPTION OF THE INVENTION

These and further objects and relative advantages, which will be better clarified in the following description, are achieved by a process for the identification of plant species and varieties, identifiable both individually and in a mixture via the use of probes.

The process according to the present invention allows the selective identification of plant species and/or varieties, both individually and in a mixture in a given sample.

The process according to the present invention is based on the performance of a molecular analysis which highlights in an exclusive, specific and selective manner individual components of any plant sample.

The process is based on the production of diagnostic genetic fragments of precise plant species and varieties, which are thus descriptors of a barcode, a DNA-based barcode. The diagnostic fragments indicated as probes can be used individually or in a mixture thereof.

The process according to the invention allows identification of all the plant components of a mixture and has the advantage of being extremely versatile.

The process of the invention is based on the existence, extended to all plants, of specific non-codifying intron sequences, the position of which in the plant genome is highly conserved among species.

It is known that the genomes of plants belonging to the same species share parts with conserved nucleotide sequences. The genome parts with this characteristic, which could therefore be defined as the identification barcode of the species, also appear to be contained in the intron regions of some genes. These conserved regions, however, are not structured in a recognisable way, hence their identification is highly complicated and impossible to perform.

It is known in fact that the genes that codify for the protein β-tubulin, a monomer essential for assembly of the microtubules, have a genomic structure organised and maintained and conserved inter and intra species. The genes that make up the β tubulin gene family are organised according to a structure defined by 3 codifying exon sequences and two non-codifying intron sequences. This structure is conserved in all plant species, but while the exons are identical in length and have a low variability in the nucleotide sequence, the non-codifying intron sequences are highly polymorphic in length and sequence. These reciprocal characteristics had previously obstructed the development of diagnostic strategies.

The process subject of the invention overcomes the limits of the known art, since it uses the structural and sequential specificities of β-tubulin genes for recognition of the variable zones which become identifiers of the plant species or varieties. The process makes use of probes selective for the plant varieties or species. The process is extremely versatile since it can be successfully applied to identification of all plant species, even when no genomic or molecular information is available on them a priori.

The process subject of the invention overcomes many problems and limits posed by the few known processes available, such as the laboriousness, high costs, the need for expensive and sophisticated instruments, the complexity of the diagnostic data generated, the production and use of probes which are specific only to a single species or variety and therefore not able to be successfully applied to mixtures of species and/or varieties and to plants whose genome has not yet been characterised.

The process according to the invention overcomes the limits of the known art because it has proved to be easy to perform, rapid, inexpensive, highly specific, accurate, reproducible, applicable to mixtures of plant species and to plant species for which no genomic information exists, widely implementable and inexpensive.

In particular the process of the invention allows the identification and use of probes which are able to recognise the target sequence present in the samples of plant material even when the non proof-reading DNA-polymerase elongation enzyme is used in the reaction mixture for amplification via PCR (DNA-polymerase chain reaction). This increases the recognition spectrum of the target sequences which are the introns of β tubulin genes.

The basic characteristic of the probes according to the invention is their high recognition specificity of the intron regions of β tubulin which are characteristics of the species and/or variety towards which they are directed. Since the target regions in the genome are those contained in the introns, i.e. highly polymorphic in sequence and in length, the use of a non proof-reading DNA-polymerase elongation enzyme, i.e. a polymerase that does not have a 5′-3′ exonuclease activity able to correct any errors (proof reading) of incorporation of the nucleotides in the DNA strand during elongation of the strand in the PCR amplification phase of the DNA extracted from the samples, has proved to be surprisingly advantageous in providing a positive interaction of the probes with the target sequences of specific species or varieties. In this way, the probe of interest is able to anneal to the target sequence (DNA barcode), which will therefore be amplified without errors, even when isolated single base changes exist at the origin.

Thus, according to one of its embodiments, the invention concerns a process which comprises
a) Extraction of genomic DNA (gDNA) from samples
b) Selective amplification of the intron sequences of β-tubulin genes
c) Isolation of the specific bands of interest (intron regions of β-tubulin)
d) Cloning and sequencing of the intron regions of interest
e) Study and analysis of the sequences and design of the primers (understood as PROBES) specific for the sequence isolated (creation of a battery of primers)

Thus, the invention also concerns the probes able to selectively recognise the sequences of the introns of β tubulin which act as DNA barcode.

The expression “probe”, “primer”, “diagnostic genetic fragment” here indicates the specific nucleotide sequence able to selectively recognise the parts of conserved intron regions of plant species and varieties.

More specifically, the expression “probe” here indicates the specific nucleotide sequence able to recognise the parts of conserved intron regions within the single species, or the intron regions of β tubulin, used as a primer to trigger the PCR reaction.

The probe according to the invention is a nucleotide sequence used as a primer (Forward and Reverse) for triggering the reaction and has the following characteristics: it is specific for the analysed species, but universal for the single plant varieties that compose it.

The nucleotide length of the probe according to the invention is between 16 and 30, preferably 18-28, even more preferably 20-24 pairs of bases.

Therefore, for example, if we wish to verify the presence of a species like maize, it will be possible to use the process according to the invention and fine-tune at least one probe designed for the maize species which will be specific for the maize but suitable for all the plant varieties of maize.

According to a preferred embodiment of the invention, the probe is included among those contained in Table 1, which shows the selective Forward (F) and Reverse (R) probes for the species indicated.

The choice of the probe understood as a primer to be used for identification of the plant species or variety constitutes a fundamental aspect. Since the probe is used as a primer in the PCR reaction, it must be able to hybridize in a specific and efficient manner with the sequence of interest, disregarding those that are aspecific. The probes according to the invention are produced evaluating the following aspects:
percentage content of purine and pyrimidine nucleotide bases, more specifically the content of the nucleotides guanine and cytosine between 45% and 50%, decisive factors for establishing the annealing temperature of the probes
the absence of internal sequences complementary to one another or repeated inverted (palindromic) sequences to avoid the formation of primer aggregates (called primer dimers) or hairpin structures
the concentration with which the primers are commonly used within the reaction mixture for triggering the PCR. Too high a concentration of primer could lead to the amplification of non-specific sequences, whereas too low a presence of primer makes the PCR ineffective.

A further subject of the present invention is use of the probes taken individually or in a mixture thereof or differently combined. According to a preferred embodiment of the invention, the probes that can be used individually or in a combination thereof are among those described in Table 1.

A further subject of the present invention is the use of at least one probe, or a combination of probes, for selective identification and traceability of plant species and/or varieties, present individually and/or in composite mixtures and/or in combination with products of plant and non-plant origin.

The probes subject of the invention can be used for the preparation of a kit for identification of the plant species and varieties.

According to a further embodiment, the invention therefore concerns a kit for identification of the plant species and varieties which comprises the probes according to the invention.

According to a preferred embodiment of the invention, the kit for identification of the plant species and varieties comprises probes able to selectively recognise the intron regions of β-tubulin.

According to a preferred embodiment of the invention, the invention also concerns the diagnostic kit for selective identification and traceability of plant species and/or varieties, present individually and/or in composite mixtures and/or in combination with products of plant and non-plant origin in a given sample which comprises at least one single probe, or a combination thereof.

According to a preferred embodiment of the invention, the kit comprises specific probes for the recognition of different plant species, for example those indicated in table 1.

According to the present invention, “genomic DNA” (gDNA) means the total DNA extracted from the starting material, whether it is the single plant reference species or variety or whether it is the material, product or mixture of which we wish to analyse the composition in species.

According to a preferred embodiment, the invention therefore concerns a process which comprises:
a) Extraction of genomic DNA (gDNA) from samples
b) Selective amplification of the intron sequences of β-tubulin genes
c) Isolation of the specific bands of interest (intron regions of β-tubulin)
d) Cloning and sequencing of the intron regions of interest
e) Study and analysis of the sequence and design of the probes (understood as primers) specific for the isolated sequence

Optionally, the process can comprise steps f) verification of the specific amplification of the intron regions of β-tubulin genes (fine-tuning of the probe or validation of the probe) and g) creation of a battery of probes (primers). According to the present invention, step f) can also be understood as a probe validation phase. According to the present invention the term “sample” indicates a mixture, a finished or semi-finished product, or a preparation, for human or animal consumption, which can undergo the process of the invention and for which the presence or absence of plant species and/or varieties can be evaluated.

According to a preferred embodiment of the invention, the process therefore comprises:
a) Extraction of genomic DNA (gDNA) from samples, for example, from samples of plant material, obtained from any tissue of the species of interest and according to the known techniques, for example using the “classic” extraction protocols or commercial kits. In both cases, the initial step requires pulverisation of the plant material in liquid nitrogen. The quality and quantity of the gDNA extracted are evaluated both spectrophotometrically and via agarose gel electrophoresis and comparison with known quantities of double-stranded DNA extracted from the lambda phage (,DNA). Extraction of the DNA and its dosing can therefore be performed according to the known techniques. Some technical details are nevertheless provided in the experimental section of the present description.
b) Selective amplification of the intron sequences of β-tubulin genes. The intron sequences are amplified via PCR starting from quantities of gDNA preferably between 20 and 30 ng and even more preferably according to the following protocol:
   1. 3 minutes at 94° C. (initial denaturation)
   2. 14 cycles programmed as follows: 30 seconds at 94° C. (denaturation), 45 seconds at 65° C. (annealing) and reducing the annealing temperature by 0.5° C. at each forward cycle of the reaction, 2 minutes at 72° C. (extension)
   3. 15 cycles programmed as follows: 30 seconds at 94° C. (denaturation), 45 seconds at 57° C. (annealing), 2 minutes at 72° C. (extension)
   4. 8 minutes at 72° C. (final extension)
   5. keeping at 10° C. if necessary before proceeding to the next step
Some details of preferred embodiments of the invention relative to step b) and the composition of the mixture are provided in the experimental section of the present description.
c) Isolation of the specific bands of interest (intron regions of β-tubulin genes). The products obtained from the previous stage b) of PCR are separated by electrophoresis in agarose gel (or alternatively in polyacrylamide gel (PAG)) and the single fragments are displayed in the UV transilluminator (or in visible light in the case of PAG). Purification of the products of interest from agarose or polyacrylamide can be performed according to the known techniques. Some details of preferred embodiments of the invention are provided in the experimental section of the present description.
d) Cloning and sequencing of the intron regions of interest performed according to the known techniques. Some details of preferred embodiments of the invention are provided in the experimental section of the present description.
e) Study and analysis of the sequence and design of the primers (understood as PROBES) specific for the isolated sequence. In this case the following strategy is applied which comprises:
   1. comparison of the isolated sequences with those deposited in the main international databases such as

GenBank of the National Center for Biotechnology Information (NCBI) and EBI of the European Bioinformatic Center
2. recognition of the isolated sequence and design of the probes for diagnostic purposes via comparison of the terminal parts (5′-3′) of the isolated (or exon) sequences. The comparison can be direct if the exon sequence is available in the database, or indirect if no information exists. By indirect we mean a comparison performed between the exon parts of the sequence obtained according to the process of the invention and those of homologous sequences of other species already deposited in the database. The inventors have surprisingly found that particular sequences in the intron regions of β-tubulin genes are conserved in a species, as in the single varieties that compose them. The inventors have therefore found that in the intron regions of β-tubulin genes at least one DNA sequence is present which can be used as DNA barcode characterising a species and the numerous varieties pertaining to it. Therefore, for example, a nucleotide sequence identified for the wheat species identifies only the species of interest and the plant varieties of the wheat, but not those of the barley species. The presence of DNA barcode sequences has permitted the development of the surprising process according to the invention, and likewise the design, characterisation and use of the probes, again according to the present invention. Via this type of analysis it has been possible to confirm that all the sequences isolated according to the procedure described above are sequences of β-tubulin. A fundamental prerogative of the method is the capacity to amplify the same type of sequence, or introns of plant β-tubulin genes. Once it has been ascertained that the isolated sequences correspond to the sequences of the first intron of the various β-tubulin isoforms of the species in question, the single sequence is studied in order to determine the most suitable region for the design of specific primers (probes). As already indicated above, the design of the probes according to the process allows numerous technical problems to be overcome, connected with the complementarity of said probes with the isolated intron regions. In fact, the intron regions often present repeated strings rich in thymine (T) and adenine (A); or strings that are repeated, almost identical, many times within the same intron; or the intron sequences within the same species differ only in length, as they present one more or one less nucleotide part and can have a high degree of homology with intron sequences of species very close to one another in evolution terms. These problems are particularly crucial, but the probes according to the present invention enable the above-mentioned limits and difficulties to be overcome.

Optionally the process can comprise steps f) verification of the specific amplification of the intron regions of β-tubulin genes (fine-tuning of the probe) and g) creation of a battery of probes (primers).

The optional step f) comprises validation of the probe via a PCR reaction. It may be advantageous to optimise the amplification protocol, or the number and duration of the cycles and the annealing temperature of the probes in order to obtain one single amplified product in a satisfactory quantity.

According to the probe considered, basically two amplification protocols are used (A or B). For these, the optimised variables (indicated in Table 2) concern mainly the duration of the extension phase (EX), the number of cycles (CN) and the annealing temperature of the primers (AT).

$$\begin{matrix}1. 3 minutes at 94 ° C . ( initial denaturation ) \\ 2. CN \{ \begin{matrix}30 seconds at 94 ° C . ( denaturation ) \\ EX ( in seconds ) at AT ( annealing ) \\ 30 seconds at 72 ° C . ( extension )\end{matrix} \\ 3. 1 minute at 72 ° C . ( final extension ) \\ 4. keeping at 10 ° C . & Amplification protocol A \\ 1. 5 minutes at 72 ° C . ( initial denaturation ) \\ 2. CN \{ \begin{matrix}30 seconds at 94 ° C . ( denaturation ) \\ EX ( in seconds ) at AT ( \begin{matrix}annealing + \\ extension\end{matrix} )\end{matrix} \\ 3. 3 minute at 72 ° C . ( final extension ) \\ 4. keeping at 10 ° C . & Amplification protocol B\end{matrix}$$

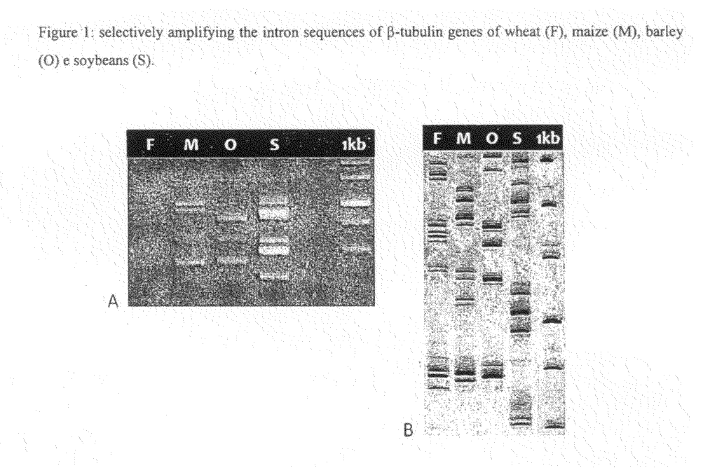

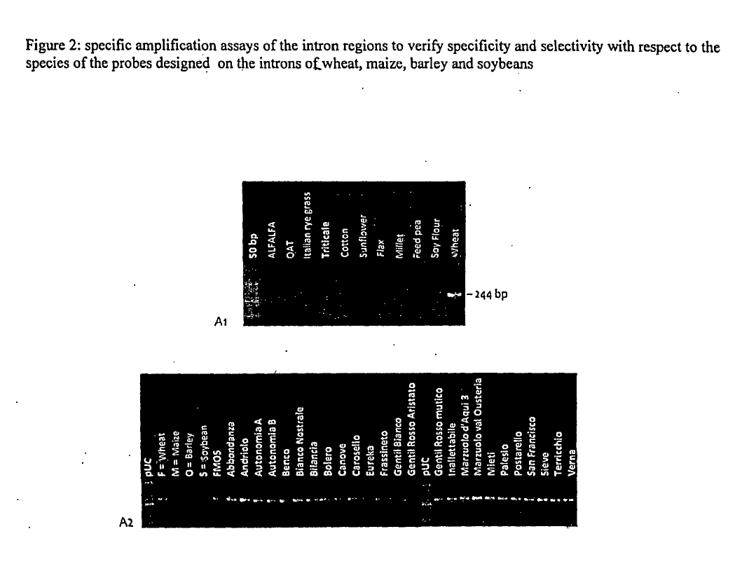

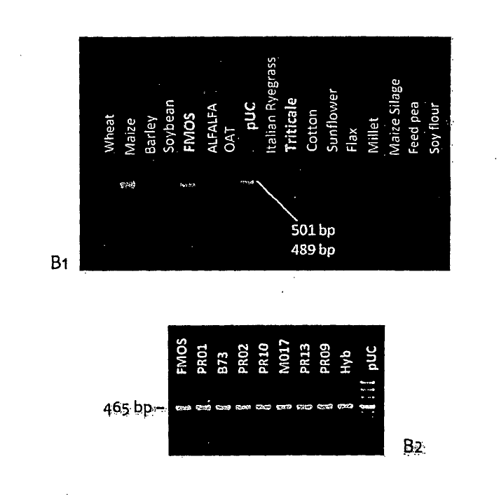

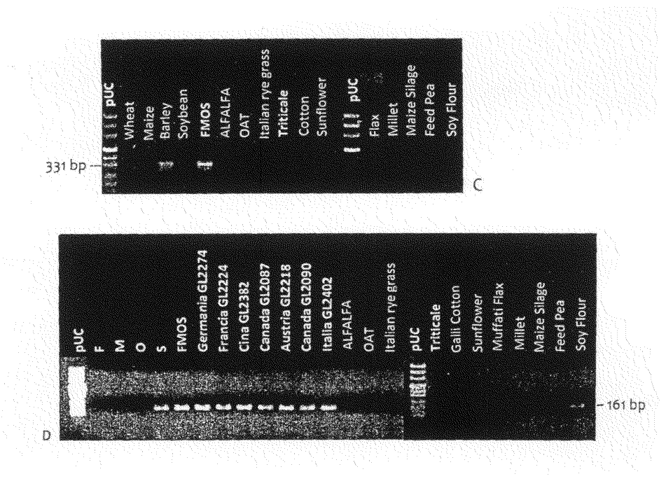

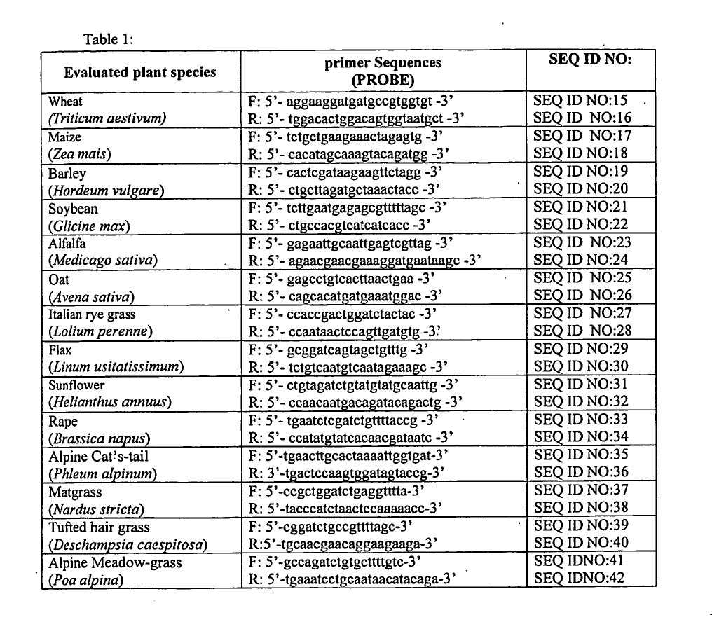

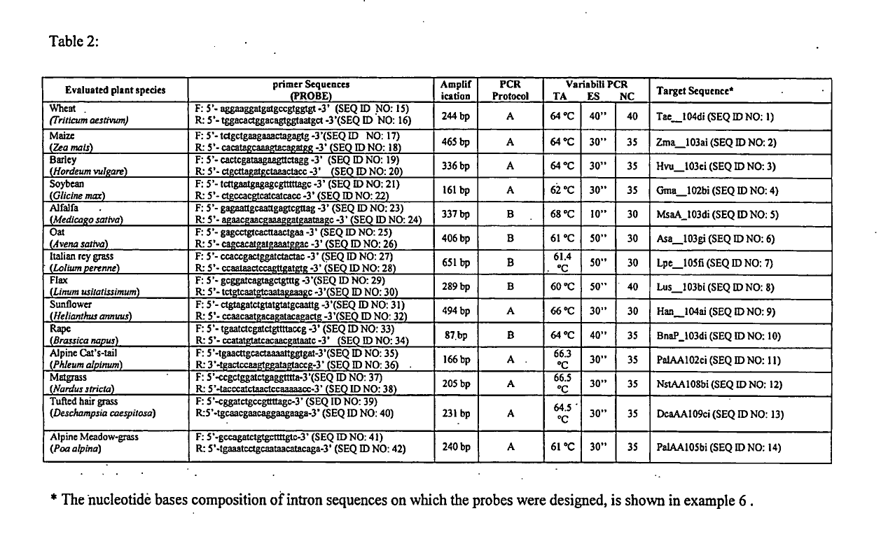

Some details of preferred embodiments of the invention are provided in the following experimental section, which refers to the attached FIGS. 1 and 2 which show:

(FIG. 1) the results of the selective amplification of the intron sequences of β-tubulin genes of wheat (F), maize (M), barley (O) and soybean (S). The gels show the products of the PCR reactions separated by electrophoresis in agarose gel (A) or polyacrylamide gel (B). On the right, a molecular weight marker has been loaded. The single bands have been isolated from the agarose or polyacrylamide matrix and purified, cloned and sequenced.
(FIG. 2) the results of assays of specific amplification of the intron regions of β-tubulin genes of DNA which verify the specificity and selectivity of the probes with respect to the species. The gels show the validation of some probes:
   a) for the WHEAT the probe designed on the intron sequence Tae_{—}104 of (SEQ ID NO: 1) permits amplification of a fragment of 244 bp. The sample FMOS was extracted from a mixture of the four species (wheat, maize, barley and soybean) prepared in the laboratory. The probe was validated on different plant species (A1) and on different varieties of soft wheat (A2).
   b) for the MAIZE the probe designed on the intron sequence Zma_{—}103ai (SEQ ID NO: 2) allows amplification of a fragment of 465 bp. The probe was validated in different plant species (B1) and on different varieties of maize (B2).
   c) for the BARLEY the probe designed on the intron sequence Hvu_{—}103ei (SEQ ID NO: 3) allows amplification of a fragment of 336 bp. The probe was validated on different plant species.
   d) for the SOYBEAN the probe designed on the intron sequence Gma_{—}102bi (SEQ ID NO: 4) allows amplification of a fragment of 161 bp. The probe was validated on the wheat (F), maize (M), barley (O), soybean (S) species and on a mixture of them (FMOS), on different varieties of soybean (from GL2274 to GL2402) and on other plant species.

### Experimental Section

### EXAMPLE 1

### Extraction of Genomic DNA (gDNA) from Plant Material and Selective Amplification of the Intron Sequences of β-Tubulin Genes

The gDNA can be extracted from any tissue of the species of interest, using traditional protocols or commercial kits, for example those currently known commercially as GenElute Plant Genomic DNA miniprep kit (Sigma-Aldrich S.r.l.). In both cases the initial step requires pulverisation of the plant material in liquid nitrogen. The quality and quantity of the extracted gDNA are evaluated both spectrophotometrically and via agarose gel electrophoresis and comparison with known quantities of double-stranded DNA of the lambda phage (λDNA).

The intron sequences are then amplified via PCR (Polymerase Chain Reaction) starting from 20-30 ng of gDNA. The reaction mixture (in a volume of 30 μl) contains: 1U Taq Polymerase (non proof-reading) and the required quantity of buffer associated with it, 1.5 mM Magnesium Oxalacetate, 0.52 mM dNTPs (equimolar mixture of deoxynucleotides), 2 μM trigger oligonucleotides (Forward:5′-AACTGGGCBAARGGNCAYTAYAC-3′ (SEQ ID NO: 43); Reverse:5′ACCATRCAYTCRTCDGCRTTYTC-3′ (SEQ ID NO: 44)). At the level of β-tubulin genes, the trigger sequences are positioned at the end 3′ of the first exon and at the 5′ of the second. In this way the PCR products are given by the sequences of the first intron, flanked by small portions of the first (at the 5′) and second (at the 3′) exon, of all β-tubulin genes of the species in question. Amplification was conducted according to the following protocol:
1. 3 minutes at 94° C. (initial denaturation)
2. 14 cycles programmed as follows: 30 seconds at 94° C. (denaturation)
   45 seconds at 65° C. (annealing)
   2 minutes at 72° C. (extension)
(at each cycle the annealing temperature drops by 0.5° C.)
3. 15 cycles programmed as follows: 30 seconds at 94° C. (denaturation)
   45 seconds at 57° C. (annealing)
   2 minutes at 72° C. (extension)
4. 8 minutes at 72° C. (final extension)
5. keeping at 10° C. if necessary before proceeding to the next stage

### EXAMPLE 2

### Isolation of the Specific Bands (Intron Regions of β-Tubulin Genes)

The products amplified by the PCR are separated via electrophoresis in agarose gel (or alternatively in polyacrylamide gel, PAG) and the single fragments, displayed in the UV transilluminator due to the bond with ethidium bromide (or in visible light with silver nitrate in the case of PAG), are excised from the gel with the aid of a scalpel. To purify the DNA fragment of the agarose cube, or polyacrylamide strip, various commercial kits can be used.

### EXAMPLE 3

### Cloning and Sequencing of the Intron Regions of β-Tubulin

Via the use of commercial kits, for example those currently known commercially as TOPO TA Cloning (Invitrogen S.R.L.), the individual intron sequences obtained for example as reported in the preceding example, are inserted in plasmid vectors for transformation into bacteria. The strain of competent bacterial cells, usually E. coli, is usually supplied with the kit and the transformation procedure is performed via thermal shock according to the directions of the manufacturer. Selective growth on LB agar plates in the presence of the appropriate antibiotic allows the single transformed bacterial colonies to be identified and isolated.

The transformants are selected by means of a PCR reaction according to the directions of the kit manufacturer. The positive clones are inoculated in liquid medium and grown under stirring for the subsequent preparation of plasmid DNA, which occurs by alkaline lysis according to traditional protocols. The plasmids thus obtained are prepared for the sequencing and sequenced on both the strands. This procedure can be applied to any plant species and in particular is shared for the 14 different plant species listed in Table 1.

### EXAMPLE 4

### Study of the Sequence and Design of the Primers (Probes) Specific for the Isolated Sequence (Creation of a Battery of Primers)

A first analysis entails comparison of the sequences isolated with those deposited in the main international databases. The fundamental prerogative of the method is the ability to amplify the same type of sequence, introns of plant β-tubulin genes, from genomes already sequenced or not. The recognition of the specificity of the isolated sequence and the subsequent design of the probes for diagnostic purposes use the comparison performed only on the terminal parts (at 5′ and at 3′) of the isolated sequences, or the exon sequences. The comparison is direct, if the sequence of the exons is available in the database, or indirect if no information exists. By indirect we mean a comparison performed between the exon parts of the sequence obtained and those of homologous sequences of other species already deposited in the database. The indirect comparison is made only on the exon parts using sequence alignment bioinformatics programs such as BLAST provided on line by the National Institutes of Health, NIH-NCBI, Bethesda Md., USA. The program algorithm compares the sequence inserted with all those of all the organisms present in the main international databases, thus allowing verification that the sequence isolated codifies for a β-tubulin. This type of analysis has confirmed that all the sequences isolated according to the procedure described above are β-tubulin sequences. Once it has been verified that the isolated sequences correspond to the sequences of the first intron of the various isoforms of β-tubulin of the species under examination, the single sequence is then studied in order to determine the most suitable region for the design of specific primers (probes).

The inventors have thus surprisingly found that particular sequences in the intron regions of β-tubulin genes are conserved in a species, as in the single varieties that compose it. The inventors have therefore ascertained that in the intron regions of β-tubulin genes, at least one DNA sequence is present which can be used as DNA barcode that characterises a species and the numerous varieties which can pertain to it. Therefore, for example, a nucleotide sequence identified for the wheat species identifies only the species of interest and the plant varieties of wheat, but not of the barley species. The presence of DNA barcode sequences has permitted development of the surprising process according to the invention, and definition of the design, characterisation and use of the probes, again according to the present invention.

### EXAMPLE 5

### Assays of Specific Amplification of the Intron Regions of β-Tubulin (Probe Validation)

The probe is validated by means of a PCR reaction. The standard reaction mixture (in a volume of 20 μl) contains: 20-30 ng gDNA, 0.66U Taq Polymerase (non proof-reading) and the required quantity of buffer associated with it, 1.5 mM Magnesium Oxalacetate, 0.52 mM dNTPs (equimolar mixture of deoxynucleotides), 1 μM primer forward (F) and 1 μM primer reverse (R). For each plant species examined, Table 2 gives by way of example the nucleotide sequence of a validated probe, with the relative optimised amplification conditions following experimental assays.

According to the probe considered, basically two amplification protocols are used (A or B). For them the optimised variables (indicated in table 2 below) concern mainly the duration of the extension stage (EX), the number of cycles (CN) and the annealing temperature of the primers (AT).

$$\begin{matrix}1. 3 minutes at 94 ° C . ( initial denaturation ) \\ 2. CN \{ \begin{matrix}30 seconds at 94 ° C . ( denaturation ) \\ EX ( in seconds ) at AT ( annealing ) \\ 30 seconds at 72 ° C . ( extension )\end{matrix} \\ 3. 1 minute at 72 ° C . ( final extension ) \\ 4. keeping at 10 ° C . & Amplification protocol A \\ 1. 5 minutes at 72 ° C . ( initial denaturation ) \\ 2. CN \{ \begin{matrix}30 seconds at 94 ° C . ( denaturation ) \\ EX ( in seconds ) at AT ( \begin{matrix}annealing + \\ extension\end{matrix} )\end{matrix} \\ 3. 3 minute at 72 ° C . ( final extension ) \\ 4. keeping at 10 ° C . & Amplification protocol B\end{matrix}$$

The diagnostic specificity of each individual probe is verified, again via PCR and with the above conditions, on different species and, when available, on different varieties of the same species. In the first case selective amplification is observed only in the plant species for which the probe has been isolated. In the second case an amplification signal is observed common to all the varieties belonging to the plant species for which the probe is selective.

The PCR products are separated by agarose gel electrophoresis and the single fragments are displayed in the UV transilluminator due to the bond with the ethidium bromide.

Table 2, a variation of Table 1, presents additional data relative to the dimension of the amplified product, the PCR protocol and its execution variables, and the reference to the target sequence of the probe according to the invention. Table 2 summarises example 5.

### EXAMPLE 6

### Isolated Intron Sequences

The isolated intron sequences of β-tubulin are given below (target sequences indicated in Table 2) on which the probes were designed:

>Tae_104di
(SEQ ID NO: 1)
GTAACTGAACTTGCACAAATTGGCGATAGTTTATAAGCCTTATGACAGGC
TTTACTTCTATTAAGTTGAGTTTGGGTATAAGAATGCATTATTTCATGGT
ATACAGATTTGAATTATGTTGTCACACAATAAGAAGTTTTAGGCTAGAAT
ATGTATTATGCTAGTAATGCTATGTGATGATGCCGTGGCATCTTCTGTTG
GTATGGAATCTTTTTGTATAGCCATGATTACTTGTTTGTGAATAAGGGCA
ATGGTTGTCAGGAAGGATGATGCCGTGGTGTATGATGATTGAGAGTTAGT
ACTAGGTCTCTGCTTTCATTGTTCTGTATTGTTGTCATGACTCTCATTGC
TTCCATATCTTGTGAATTATTCTGATAGTGAGACATCTAGCATAACTACT
GATTATGTACCTATGCTGCTTTCATCTATTTGTGTCTAAATAGTTGTTAT
CTATTCAGGGTGGTTATCTATTTATATTCAGCATTACCACTGTCCAGTGT
CCACTTGAAATCATTATTTGAGCATCTTTTATGTTTTCAG
>Zma_103ai
(SEQ ID NO: 2)
GTACTTGAGCTTATTGATGATAAAGCTGATTGCTTCAAGTTTTTATATTT
GTTTCTGCTGAAGAAACTAGAGTGAGATGTTCATGTTGAAGTATCTCTAC
AGTTTCTTTACCCATGCTTGTGATTTTTTTCACTACAATGCGATACATTG
AATCCATATAGGGCTGAAGCTATCCATGGAACTAGAACTGCGATATCCTG
TTATAATGAAGCTGCTTTTGCTCTAGAACCTAAGTCATTTCACTCGTTTA
GTTCATGTCCTATAATATAGAATGGATTATCCTAAATGACACTGATACTT
ATTGGTCCTTGCCGATTGCTTAGTATTTTCACATTTGAGTATCTTTTGTG
CTGTTGCCATGTTTTGACACATGAGCAGAAACAATTTTTTTACTTGCACT
TGTTGCTTGAACAATTGGCATACCAAAATACTGTACATAAAAAAGCTGTT
CAGACTGTGATTACTAAATCACCTATGCGTGTCATGTATCTTGGTTGCCA
TCTGTACTTTGCTATGTGTTTTTCCTAATCACCCAAGTAACTAAACTTAA
TTATCATGCATTGAACATTAACTCAATCTATTTGTTTATTCAG
>Hvu_103ei
(SEQ ID NO: 3)
GTAACTGAACTTGAATTGAATTATTTCGTATATGCAGATTTGAATTATGT
TGTCACTCGATAAGAAGTTCTAGGCTACAATATGTATTATGCCAGCAATG
CTATGCGATGCTGCTGTGGCATCTTCTGTTTGTATCCTTCTTATTTATAG
CCATGATTACTTGTTTGTAAATAAGGGTACTGGTTGGAAACCCTACGTAC
TTGGATAACTTTGTGTATGATCATTCAGAGTTAGTACTAGGTCTCTGCTT
ACATTGTTTTGTATTCTTGTCATGGCACTTGTTACTTCCGTATCTGGTGA
ATTATTTCATAGTGCCATCTAGCATAACTAATGATTATGTATCTATGCTG
CTTTCGTAGAATATATTTGGTAGTTTAGCATCTAAGCAGTGGTTATCTAT
TTATGTTCAGCATTACCACTATACACTTCAAATCATTGTTTAGAAGAATC
TTTTTATGTTTTCAG
>Gma_102bi
(SEQ ID NO: 4)
GTATAACCAATTAGAATAACTATCTTGAATGAGAGCGTTTTTAGCTGTCA
TTCTTCAACTAAAGTATTTTTCGATCTATGTACATTGCGTAAGTTAAATC
TACTGTTTGGTTACTAATTTTGATTGATTTGATTGGATTGTTGTGGTTTT
GATGGGATTGGAGTGGTGATGATGACGTGGCAG
>MsaA_103di
(SEQ ID NO: 5)
AAGTTTGTTTATAGATTCATCGTTTTTGTTTTTAGTAAGGTTGAATTTGT
GTTGTTTTGAGAGAATTGCAATTGAGTCGTTAGATAATTGCGATGTTGAA
TTGCGGTTGTGTCGTTTTATATTGTGAGTTTGTTCGCAGGTTCATTATTT
TCATTTTTAGTATTTAGTGATGTTGAATTTGTGTTGTATTTTATGTGTTC
TATTCGATCTGAATGAGTGAATATCGTTTTTTAGTAGCAAATATTAAATG
ATTAAATGATCGTTTTTAGCGATGTTGAATATCATTTTTTGGTGATGTTG
AACTGGTGTTGTTTTCGAGAAAATTATGATTGCTTGCAAGGTAATTTTTC
CGTTTTATGTTGCGAGTTTTTTTGCTTATTCATCCTTTCGTTCGTTCTTA
GCAATGTTGAATTTGTGTTGTTTTCGATGAATTTCACCGATTGGAATGAA
TTAATAGTATTATTTGGTAGATAAATTTAAATGATTATGAATTGTTATGG
ATTTGTTATGATTTTT
>Asa_103gi
(SEQ ID NO: 6)
TAAAGAGCCTGTCACTTAACTGAATTCTGACAACTGCAAGATCGATTTTT
TTTTCTTCAGTGACAACTGCTAGATCGATTGTGCATGCTTGAAGCTGGCC
TGTGTTTCGTTACTTGTATATCATGTTTAAGATGTCCAGCACACATTTTA
TTATCCATGATATGGGGTGCAGCCAATACTAAGCCTTGGGCGATACTGTT
CTTCAGATTTGTTGGCCAACTAATTTGTTAGCATTACAAGACTTCTAACA
CAGTAAACATTCTTCTGAGATAATATAGCGGTTAACAAGGCTTGCATAAT
CCACCTGTTGGAAATTCAGTGTTCTTGTATTGTTTTCTAGTTGGAAGTGT
ACTGAATTTATATGTTTTCCTTTATTTTTCAAGTATATAGTCCATTTCAT
CATGTGCTGATAATAATTAATTCCTTATGTTTTTGTTTTC
>Lpe_105fi
(SEQ ID NO: 7)
TATAACCACCGACTGGATCTACTACTTGTTTTGCCGGAAATTTTACTCCT
CAGATCTCGTAAGGTTTTGGTGGGGCATGTAGATCTAGTTCCCCATCCGT
GGTAGAACGCTGCGATTTTAGGGTTCTCGTCTCCTAGATCTGGCCGTTCC
ACCTCCTTTTCCGTCGTAGGTGGTTCTGGACTTCTAGATCTGGCTTGCGG
TAGCTGTCGGAACATGAATCATTCTTCATTCTACTTGTGCTGTTGTTGCT
GGGGTTAACATTTGGGATGGTGTTATGCGGTTCTAACTGCCGAGATCCGT
CGAATTGATGTGGTTGCTGTCTACAGCTTTGCAGATTTCCTCACTCACTT
TGTTCATTCTACTTGTGTTGCTGTTGTTGGGGGGTGACATTTTGGATTGT
GTATTATAGATACAGGCATGCGTTCCTTTGGCTGCAAAAACATGATGGAT
ATCTCTTCACTCATCACTGCTCTTTGTAAAGTAGAGTAAAATATGGCTGA
AGATTAAAGCATCACCAGGGGAGTAGCATGCCTGATGTTGATCCCTCAAG
CACCCAGTGCTTACGATTCTGCTCAATGCATTCATTGTATTGCAATTTGT
TATATGCGTGTGTAGATTGCTTAACGTTTGAAAACACATCAACTGGAGTT
ATTGGGTATCCTTTTTTTATTCAGTTGCCAACTGAAAATGAGATCCAAAT
AACTTATATGCTTCTATTTACCAC
>Lus_103bi
(SEQ ID NO: 8)
GTAGTTTTTCTCTTCTTTTTCTCTGCTGCGGATCAGTAGCTGTTTGGCTA
GAAGTGTCGATTGATTGATTTTCAAGTTACTTCGTTAATTTTACTTTGAT
TATTGAGCACTTGCATTTTGTTTACTCAATCACTCTGCACAGAATTCCAA
GTCAATTTTACTAAGAACAGTTCAAAGCGGCATTGATGAACGGTTCTAGT
GTTGAATTATACCTGTTGACATTAGATGGAAGAGTTTCGTGATCTAGAAT
GTTTGTGATCATACATCTTGGATATGATTCTTAGTTAACGCTATGCTTTC
TATTGACATTGACAGATTGTTATGTCACCTTAGAATTTGTAGTACTTTGG
TCCTTAATCATCTTACTAATCAGTCTCTCGATTCTT
>Han_104ai
(SEQ ID NO: 9)
TTACGGGGTATGTATTCGCATCTATTGGTGTCATGATTTGGTTTAGAATA
GTGTTAGAACTAACAGATCTAAGTTTTATTTTGCGTTGACGGTTATGTTT
ACATTTTGTTGTTTGTTTTGAAGGTATATATGTGAGTTTTAGTAAGATTT
GTTCTGTAGATCTGTATGTATGCAATTGATGATATTGTGTAGAATGTATG
TAGATCTGTAACCGTTTGTTGATATTTTACAGAACGCGTGTGAAACAAGT
GATGGTTAGTGCATTAGCTAGTTTGTACTGTTCTGTTTTGAAATGTTGTG
CTAGAAATGCTAGATCCTGCATCTTTTGTGTATTGATTGTCGATTATGTC
CAAGTCACGAGACCGTCTAGATTTGTTTTATTTTGAATAATATTGTAGAT
CTGCAACCGTTTATTTATTTTTCACTAGAGCACACGTGAAGCAAGTGATA
GTTGTTTTATTAGTTAATTTGCTTTTGAATTCTTTTGGTAGAAATGCTGT
TATTGCATTTTTCATGTATTAAATACTGATTGTAGAGTATATGCAGTTGA
CTAACAGTCCAGATTTGGATATTTTTTTTGATCTGCAAACTGTTGATTGA
TTTTCATTAGATGGTAGTTTATTACAGTCTGTATCTGTCATTGTTGGGTT
AGAAATTCTAGACTTACTTTCGGTGTTGGTTCTTAAGGTTTTATATTTAT
ATTTGTTTATG
>BnaP_103di
(SEQ ID NO: 10)
GAGAAGATAACTGATTTTTGTTTTTGAATCTCGATCTGTTTTACCGTCGT
GCGTTGATTTGATTTGAGATCTCTGTGATGTATTGATGCGATTATCGTTG
TGATACATATGGTTTTTGATCGTTGTGT
>PalAA102ci
(SEQ ID NO: 11)
GTAACTGAACTTGCACTAAAATTGGTGATAGTATAGGATCTGCACTTGTT
AAATTGAATTTACATTAGTTCATAATGCACAGATTTGTATTGTGTGTTCA
TGCCTGTGTAGTTTTTGGCTCCAAATGCCTCTCTTATAATATCTAGTATC
GGTACTATCCACTTGGAGTCATTATTAATGCTTGTGTCTTTTCAG
>NstAA108bi
(SEQ ID NO: 12)
GTACGTGTGTAGCCCGCTGGATCTGAGGTTTTATCCTGTTTCTGTTCGAT
TTGTGGAGGTGTGTGGCTTGTAGATCTAGGTTTCATGCTGCATTGTATGC
ATCCTGAGCTTGTAGATCTGAACATTTTGGACCAGATTTAACCGATATTT
AGGACTGAGGCGGCCAATTCTCATGTTTTTGCTTGGTCCTTACTAGTGGT
TTTTGGAGTTAGATGGGTAGTAATTATCTCTCTATTCACTGCAG
>DcaAA109ci
(SEQ ID NO: 13)
GTACGTCCCCGCCGGATCTGCCGTTTTAGCCTCTCTCTGTCCGATTACCA
GAGCCACATGGCTTGCAGATCTAGCTTTTATGCCCGACAGATTTTTATGT
CCGAGTTCTAGATCTGGAATGTTTGCCTAGATCTAACCGATCTACGGGAC
CATTTCGGGAGTAAACGGTTAATTTCTAGCAGTTTTTATGCAAGCTGCGG
AGTTGGATGTGCAACTAATTATTATCTTCTTCCTGTTCGTTGCAG
>PalAA105bi
(SEQ ID NO: 14)
GTAGGTCCCGCCAGATCTGTGCTTTTGTCCTCTCTCTGTTTCGATTCCTA
GCTCCATATGGCTTGCAGATCTAGGTTTTATTCTGTATTTTTGTGTGTTA
TAAGTTCTAGATCTGAAATGTTTGGCGTAGATCTATTCGATTTGCTACTC
CGTTTAGGAATGAAACGATAATTTCTAGTGTTTTGCTAAGCAAATAGCGG
CGTAATATGTATTAGCAGTAATTATCTGTATGTTATTGCAGGATTTCAG

1. Process for the selective identification and traceability of plant species and/or varieties, present individually and/or in a mixture thereof and/or with different products in a sample, including:
a) extracting genomic DNA (gDNA) from said sample
b) selectively amplifying the intron sequences of β-tubulin genes
c) isolating the specific bands of interest (intron regions of β-tubulin)
d) cloning and sequencing the intron regions of interest
e) studying and analysing the sequence and designing the probes (to be intended as primers) specific for the isolated sequence.

2. Process according to claim 1 characterized in that said intron regions of β-tubulin are selected from anyone of the sequences comprised between SEQ ID NO: 1 and SEQ ID NO: 14, included.

3. Process according to claim 1 characterized in that said process further includes the steps of f) verifying the specific amplification of the intron regions of β-tubulin genes (fine-tuning of the probe) and g) creating a battery of probes (primers).

4. Process according to claim 1 characterized in that said stage b) of selectively amplifying the intron sequences of β-tubulin genes is carried out by PCR.

5. Process according to claim 1 characterized in that said stage b) of selectively amplifying the intron sequences of β-tubulin genes is carried out by employing non-proofreading DNA polymerase.

6. Process according to claim 2, characterized in that said stage f) of verifying amplification or stage of validating said probes is carried out by PCR.

7. Process according to claim 2, characterized in that said stage f) of verifying the specific amplification of the intron regions of β-tubulin genes (fine-tuning or validation of the probe) is selected from:
$$\begin{matrix}1. 3 minutes at 94 ° C . ( initial denaturation ) \\ 2. CN \{ \begin{matrix}30 seconds at 94 ° C . ( denaturation ) \\ EX ( in seconds ) at AT ( annealing ) \\ 30 seconds at 72 ° C . ( extension )\end{matrix} \\ 3. 1 minute at 72 ° C . ( final extension ) \\ 4. keeping at 10 ° C . & Amplification protocol A \\ 1. 5 minutes at 72 ° C . ( initial denaturation ) \\ 2. CN \{ \begin{matrix}30 seconds at 94 ° C . ( denaturation ) \\ EX ( in seconds ) at AT ( \begin{matrix}annealing + \\ extension\end{matrix} )\end{matrix} \\ 3. 3 minute at 72 ° C . ( final extension ) \\ 4. keeping at 10 ° C . & Amplification protocol B\end{matrix}$$

8. Probe for the selective recognition of parts of intron regions of the β-tubulin genes.

9. Use of a probe of claim 7, as a primer for triggering the amplification protocol selected from A or B.

10. Probe of claim 7 characterized in that it is of nucleotide length ranging from 16-30, preferably 18-28, even more preferably 20-24 (base pairs).

11. Probe of claim 6, selected from anyone of the sequences comprised between SEQ ID NO: 15 and SEQ ID NO: 42, included.

12. Use of at least one probe, or a combination of probes, for the selective identification and traceability of plant species and/or varieties, present individually and/or in composite mixtures and/or in combination with products of plant and non-plant origin.

13. Diagnostic kit for the selective identification and traceability of plant species and/or varieties, present individually and/or in composite mixtures and/or in combination with products of plant and non-plant origin in a given sample, which includes at least a single probe, or a combination of probes selected from those of claim 10.


SEQUENCE LISTING (44 sequences)

SEQ ID NO: 1 - DNA, 540 bp, Triticum aestivum
  Intron (1)..(540)
gtaactgaacttgcacaaattggcgatagtttataagccttatgacaggctttacttctattaagttgagtttgggtataagaatgcattatttcatggtatacagatttgaattatgttgtcacacaataagaagttttaggctagaatatgtattatgctagtaatgctatgtgatgatgccgtggcatcttctgttggtatggaatctttttgtatagccatgattacttgtttgtgaataagggcaatggttgtcaggaaggatgatgccgtggtgtatgatgattgagagttagtactaggtctctgctttcattgttctgtattgttgtcatgactctcattgcttccatatcttgtgaattattctgatagtgagacatctagcataactactgattatgtacctatgctgctttcatctatttgtgtctaaatagttgttatctattcagggtggttatctatttatattcagcattaccactgtccagtgtccacttgaaatcattatttgagcatcttttatgttttcag

SEQ ID NO: 2 - DNA, 593 bp, Zea mays
  Intron (1)..(593)
gtacttgagcttattgatgataaagctgattgcttcaagtttttatatttgtttctgctgaagaaactagagtgagatgttcatgttgaagtatctctacagtttctttacccatgcttgtgatttttttcactacaatgcgatacattgaatccatatagggctgaagctatccatggaactagaactgcgatatcctgttataatgaagctgcttttgctctagaacctaagtcatttcactcgtttagttcatgtcctataatatagaatggattatcctaaatgacactgatacttattggtccttgccgattgcttagtattttcacatttgagtatcttttgtgctgttgccatgttttgacacatgagcagaaacaatttttttacttgcacttgttgcttgaacaattggcataccaaaatactgtacataaaaaagctgttcagactgtgattactaaatcacctatgcgtgtcatgtatcttggttgccatctgtactttgctatgtgtttttcctaatcacccaagtaactaaacttaattatcatgcattgaacattaactcaatctatttgtttattcag

SEQ ID NO: 3 - DNA, 465 bp, Hordeum vulgare
  Intron (1)..(465)
gtaactgaacttgaattgaattatttcgtatatgcagatttgaattatgttgtcactcgataagaagttctaggctacaatatgtattatgccagcaatgctatgcgatgctgctgtggcatcttctgtttgtatccttcttatttatagccatgattacttgtttgtaaataagggtactggttggaaaccctacgtacttggataactttgtgtatgatcattcagagttagtactaggtctctgcttacattgttttgtattcttgtcatggcacttgttacttccgtatctggtgaattatttcatagtgccatctagcataactaatgattatgtatctatgctgctttcgtagaatatatttggtagtttagcatctaagcagtggttatctatttatgttcagcattaccactatacacttcaaatcattgtttagaagaatctttttatgttttcag

SEQ ID NO: 4 - DNA, 183 bp, Glycine max
  Intron (1)..(183)
gtataaccaattagaataactatcttgaatgagagcgtttttagctgtcattcttcaactaaagtatttttcgatctatgtacattgcgtaagttaaatctactgtttggttactaattttgattgatttgattggattgttgtggttttgatgggattggagtggtgatgatgacgtggcag

SEQ ID NO: 5 - DNA, 516 bp, Medicago sativa
  Intron (1)..(516)
aagtttgtttatagattcatcgtttttgtttttagtaaggttgaatttgtgttgttttgagagaattgcaattgagtcgttagataattgcgatgttgaattgcggttgtgtcgttttatattgtgagtttgttcgcaggttcattattttcatttttagtatttagtgatgttgaatttgtgttgtattttatgtgttctattcgatctgaatgagtgaatatcgttttttagtagcaaatattaaatgattaaatgatcgtttttagcgatgttgaatatcattttttggtgatgttgaactggtgttgttttcgagaaaattatgattgcttgcaaggtaatttttccgttttatgttgcgagtttttttgcttattcatcctttcgttcgttcttagcaatgttgaatttgtgttgttttcgatgaatttcaccgattggaatgaattaatagtattatttggtagataaatttaaatgattatgaattgttatggatttgttatgattttt

SEQ ID NO: 6 - DNA, 440 bp, Avena sativa
  Intron (1)..(440)
taaagagcctgtcacttaactgaattctgacaactgcaagatcgatttttttttcttcagtgacaactgctagatcgattgtgcatgcttgaagctggcctgtgtttcgttacttgtatatcatgtttaagatgtccagcacacattttattatccatgatatggggtgcagccaatactaagccttgggcgatactgttcttcagatttgttggccaactaatttgttagcattacaagacttctaacacagtaaacattcttctgagataatatagcggttaacaaggcttgcataatccacctgttggaaattcagtgttcttgtattgttttctagttggaagtgtactgaatttatatgttttcctttatttttcaagtatatagtccatttcatcatgtgctgataataattaattccttatgtttttgttttc

SEQ ID NO: 7 - DNA, 724 bp, Lolium perenne
  Intron (1)..(724)
tataaccaccgactggatctactacttgttttgccggaaattttactcctcagatctcgtaaggttttggtggggcatgtagatctagttccccatccgtggtagaacgctgcgattttagggttctcgtctcctagatctggccgttccacctccttttccgtcgtaggtggttctggacttctagatctggcttgcggtagctgtcggaacatgaatcattcttcattctacttgtgctgttgttgctggggttaacatttgggatggtgttatgcggttctaactgccgagatccgtcgaattgatgtggttgctgtctacagctttgcagatttcctcactcactttgttcattctacttgtgttgctgttgttggggggtgacattttggattgtgtattatagatacaggcatgcgttcctttggctgcaaaaacatgatggatatctcttcactcatcactgctctttgtaaagtagagtaaaatatggctgaagattaaagcatcaccaggggagtagcatgcctgatgttgatccctcaagcacccagtgcttacgattctgctcaatgcattcattgtattgcaatttgttatatgcgtgtgtagattgcttaacgtttgaaaacacatcaactggagttattgggtatcctttttttattcagttgccaactgaaaatgagatccaaataacttatatgcttctatttaccac

SEQ ID NO: 8 - DNA, 386 bp, Linum usitatissimum
  Intron (1)..(386)
gtagtttttctcttctttttctctgctgcggatcagtagctgtttggctagaagtgtcgattgattgattttcaagttacttcgttaattttactttgattattgagcacttgcattttgtttactcaatcactctgcacagaattccaagtcaattttactaagaacagttcaaagcggcattgatgaacggttctagtgttgaattatacctgttgacattagatggaagagtttcgtgatctagaatgtttgtgatcatacatcttggatatgattcttagttaacgctatgctttctattgacattgacagattgttatgtcaccttagaatttgtagtactttggtccttaatcatcttactaatcagtctctcgattctt

SEQ ID NO: 9 - DNA, 711 bp, Helianthus annuus
  Intron (1)..(711)
ttacggggtatgtattcgcatctattggtgtcatgatttggtttagaatagtgttagaactaacagatctaagttttattttgcgttgacggttatgtttacattttgttgtttgttttgaaggtatatatgtgagttttagtaagatttgttctgtagatctgtatgtatgcaattgatgatattgtgtagaatgtatgtagatctgtaaccgtttgttgatattttacagaacgcgtgtgaaacaagtgatggttagtgcattagctagtttgtactgttctgttttgaaatgttgtgctagaaatgctagatcctgcatcttttgtgtattgattgtcgattatgtccaagtcacgagaccgtctagatttgttttattttgaataatattgtagatctgcaaccgtttatttatttttcactagagcacacgtgaagcaagtgatagttgttttattagttaatttgcttttgaattcttttggtagaaatgctgttattgcatttttcatgtattaaatactgattgtagagtatatgcagttgactaacagtccagatttggatattttttttgatctgcaaactgttgattgattttcattagatggtagtttattacagtctgtatctgtcattgttgggttagaaattctagacttactttcggtgttggttcttaaggttttatatttatatttgtttatg

SEQ ID NO: 10 - DNA, 128 bp, Brassica napus
  Intron (1)..(128)
gagaagataactgatttttgtttttgaatctcgatctgttttaccgtcgtgcgttgatttgatttgagatctctgtgatgtattgatgcgattatcgttgtgatacatatggtttttgatcgttgtgt

SEQ ID NO: 11 - DNA, 195 bp, Phleum alpinum
  Intron (1)..(195)
gtaactgaacttgcactaaaattggtgatagtataggatctgcacttgttaaattgaatttacattagttcataatgcacagatttgtattgtgtgttcatgcctgtgtagtttttggctccaaatgcctctcttataatatctagtatcggtactatccacttggagtcattattaatgcttgtgtcttttcag

SEQ ID NO: 12 - DNA, 244 bp, Nardus stricta
  Intron (1)..(244)
gtacgtgtgtagcccgctggatctgaggttttatcctgtttctgttcgatttgtggaggtgtgtggcttgtagatctaggtttcatgctgcattgtatgcatcctgagcttgtagatctgaacattttggaccagatttaaccgatatttaggactgaggcggccaattctcatgtttttgcttggtccttactagtggtttttggagttagatgggtagtaattatctctctattcactgcag

SEQ ID NO: 13 - DNA, 245 bp, Deschampsia caespitosa
  Intron (1)..(245)
gtacgtccccgccggatctgccgttttagcctctctctgtccgattaccagagccacatggcttgcagatctagcttttatgcccgacagatttttatgtccgagttctagatctggaatgtttgcctagatctaaccgatctacgggaccatttcgggagtaaacggttaatttctagcagtttttatgcaagctgcggagttggatgtgcaactaattattatcttcttcctgttcgttgcag

SEQ ID NO: 14 - DNA, 249 bp, Poa alpina
  Intron (1)..(249)
gtaggtcccgccagatctgtgcttttgtcctctctctgtttcgattcctagctccatatggcttgcagatctaggttttattctgtatttttgtgtgttataagttctagatctgaaatgtttggcgtagatctattcgatttgctactccgtttaggaatgaaacgataatttctagtgttttgctaagcaaatagcggcgtaatatgtattagcagtaattatctgtatgttattgcaggatttcag

SEQ ID NO: 15 - DNA, 22 bp, Triticum aestivum
  primer_bind (1)..(22)
aggaaggatgatgccgtggtgt

SEQ ID NO: 16 - DNA, 24 bp, Triticum aestivum
  primer_bind (1)..(24)
tggacactggacagtggtaatgct

SEQ ID NO: 17 - DNA, 21 bp, Zea mays
  primer_bind (1)..(21)
tctgctgaagaaactagagtg

SEQ ID NO: 18 - DNA, 21 bp, Zea mays
  primer_bind (1)..(21)
cacatagcaaagtacagatgg

SEQ ID NO: 19 - DNA, 21 bp, Hordeum vulgare
  primer_bind (1)..(21)
cactcgataagaagttctagg

SEQ ID NO: 20 - DNA, 21 bp, Hordeum vulgare
  primer_bind (1)..(21)
ctgcttagatgctaaactacc

SEQ ID NO: 21 - DNA, 23 bp, Glycine max
  primer_bind (1)..(23)
tcgatctatgtacattgcgtaag

SEQ ID NO: 22 - DNA, 21 bp, Glycine max
  primer_bind (1)..(21)
gtcatcatcaccactccaatc

SEQ ID NO: 23 - DNA, 23 bp, Medicago sativa
  primer_bind (1)..(23)
gagaattgcaattgagtcgttag

SEQ ID NO: 24 - DNA, 25 bp, Medicago sativa
  primer_bind (1)..(25)
agaacgaacgaaaggatgaataagc

SEQ ID NO: 25 - DNA, 20 bp, Avena sativa
  primer_bind (1)..(20)
gagcctgtcacttaactgaa

SEQ ID NO: 26 - DNA, 20 bp, Avena sativa
  primer_bind (1)..(20)
cagcacatgatgaaatggac

SEQ ID NO: 27 - DNA, 20 bp, Lolium perenne
  primer_bind (1)..(20)
ccaccgactggatctactac

SEQ ID NO: 28 - DNA, 21 bp, Lolium perenne
  primer_bind (1)..(21)
ccaataactccagttgatgtg

SEQ ID NO: 29 - DNA, 19 bp, Linum usitatissimum
  primer_bind (1)..(19)
gcggatcagtagctgtttg

SEQ ID NO: 30 - DNA, 22 bp, Linum usitatissimum
  primer_bind (1)..(22)
tctgtcaatgtcaatagaaagc

SEQ ID NO: 31 - DNA, 25 bp, Helianthus annuus
  primer_bind (1)..(25)
ctgtagatctgtatgtatgcaattg

SEQ ID NO: 32 - DNA, 23 bp, Helianthus annuus
  primer_bind (1)..(23)
ccaacaatgacagatacagactg

SEQ ID NO: 33 - DNA, 22 bp, Brassica napus
  primer_bind (1)..(22)
tgaatctcgatctgttttaccg

SEQ ID NO: 34 - DNA, 23 bp, Brassica napus
  primer_bind (1)..(23)
ccatatgtatcacaacgataatc

SEQ ID NO: 35 - DNA, 24 bp, Phleum alpinum
  primer_bind (1)..(24)
tgaacttgcactaaaattggtgat

SEQ ID NO: 36 - DNA, 22 bp, Phleum alpinum
  primer_bind (1)..(22)
tgactccaagtggatagtaccg

SEQ ID NO: 37 - DNA, 20 bp, Nardus stricta
  primer_bind (1)..(20)
ccgctggatctgaggtttta

SEQ ID NO: 38 - DNA, 22 bp, Nardus stricta
  primer_bind (1)..(22)
tacccatctaactccaaaaacc

SEQ ID NO: 39 - DNA, 18 bp, Deschampsia caespitosa
  primer_bind (1)..(18)
cggatctgccgttttagc

SEQ ID NO: 40 - DNA, 20 bp, Deschampsia caespitosa
  primer_bind (1)..(20)
tgcaacgaacaggaagaaga

SEQ ID NO: 41 - DNA, 20 bp, Poa alpina
  primer_bind (1)..(20)
gccagatctgtgcttttgtc

SEQ ID NO: 42 - DNA, 24 bp, Poa alpina
  primer_bind (1)..(24)
tgaaatcctgcaataacatacaga

SEQ ID NO: 43 - DNA, 23 bp, artificial sequence
  Forward primer used for selective amplification by PCR of the intron sequences of beta-tubulin genes according to Example 1 of the description.
aactgggcbaarggncaytayac

SEQ ID NO: 44 - DNA, 23 bp, artificial sequencce
  Reverse primer used for selective amplification by PCR of the intron sequences of beta-tubulin genes according to Example 1 of the description.
accatrcaytcrtcdgcrttytc